(19) United States
(12) Patent Application Publication
(10) Pub. No.: US 2016/0235354 A1
(43) Pub. Date: Aug. 18, 2016

(54) METHODS FOR DETECTING, MONITORING AND TREATING LYMPHEDEMA

(71) Applicant: Lymphatech, Inc., Atlanta, GA (US)

(72) Inventors: Michael J. Weiler, Atlanta, GA (US); James B. Dixon, Marietta, GA (US); Sebastian Pokutta, Atlanta, GA (US); Daniel Zink, Atlanta, GA (US); Timothy Kassis, Malden, MA (US)

(73) Assignee: Lymphatech, Inc., Atlanta, GA (US)

(21) Appl. No.: 15/041,367

(22) Filed: Feb. 11, 2016

Related U.S. Application Data
(60) Provisional application No. 62/115,374, filed on Feb. 12, 2015.

(51) Int. Cl. A61B 5/00; A61B 5/107

(52) U.S. Cl. CPC A61B 5/4312; A61B 5/0091; A61B 5/1071

(57) ABSTRACT

The present invention comprises methods for detecting, monitoring and treating lymphedema by using imaging devices to generate patient anatomical information from which body part volume measurements or geometries can be derived. Yet further, the present invention comprises methods of fitting compression garments for treatment of lymphedema by using the anatomical measurements derived from the patient anatomical information.

### CROSS-REFERENCE TO RELATED APPLICATIONS

This application claims priority to U.S. Provisional Application No. 62/115,374 filed Feb. 12, 2015. This provisional application is incorporated herein in its entirety by this reference.

### FIELD OF THE INVENTION

The present invention comprises methods for detecting, monitoring and treating lymphedema by using imaging devices to generate patient anatomical information from which body part volume measurements or geometries can be derived. Yet further, the present invention comprises methods of fitting compression garments for treatment of lymphedema by using the anatomical measurements derived from the patient anatomical information.

### BACKGROUND OF THE INVENTION

Lymphedema can occur after any cancer or treatment that affects the flow of lymph through the lymphatic vessels, such as removal of lymph nodes. It may develop within days or many years after treatment. Most lymphedema develops within three years of surgery.

Breast cancer-related lymphedema (BCRL) is a progressive, debilitating condition affecting millions of breast cancer survivors with a significant negative impact on quality of life, employment and health. BCRL is not limited to arm swelling alone. Many survivors have complex symptoms that includes breast and truncal swelling. The level of breast cancer-related arm edema can range from mild to severe, but once the condition starts, there is the possibility of progression to more severe lymphedema. Even in early stage breast cancer, various studies have shown that when lymphedema progressed, lymphedema therapy could not completely reverse it. Where lymphedema manifests, the patient is likely to suffer from lymphedema for the remainder of her life, thus leading to long-term suffering, as well as the attendant medical costs associated with the treatment of such a chronic condition. This means that prevention of lymphedema is a vastly preferred option.

In women, lymphedema occurs most frequently in the upper limbs after breast cancer surgery, in particular after axillary lymph node dissection. Essentially anyone with a breast cancer diagnosis who undergoes surgery, chemotherapy and/or radiation therapy is at relative risk for generating lymphedema. However, lymphedema can occur in any patient where the lymphatic system is altered through surgery, radiotherapy, illness and/or medication. Head and neck lymphedema can be caused by surgery or radiation therapy for tongue or throat cancer. It may also occur in the lower limbs or groin after surgery for colon, ovarian or uterine cancer, in which removal of lymph nodes or radiation therapy is required. Surgery or treatment for prostate, colon and testicular cancers may result in lymphedema, particularly when lymph nodes have been removed or damaged.

Onset of BCRL is commonly seen within the first three years following the definitive surgical procedure, with persistent, diminished risk occurring five years later and beyond. However, while risk does diminish, some patients may be anatomically predisposed to developing lymphedema. Early stage disease (Stage 1 and 2) may continue to exist in a latent or sub-clinical state even when successfully treated at initial onset, sometimes presenting at later stages ten or more years after initial diagnosis. Minor physical traumas, including cuts, burns, tight jewelry or other injuries to the fingers or hands, may transform a latent condition into active lymphedema that requires treatment. Excessive exposure to the sun can also lead to an inflammatory stimulus that overtaxes an already impaired lymphatic system, resulting in recurrent lymphedema.

Compression garments are the primary method of treatment of early stage lymphedema. Precise fitting of garments is critical for compression garments to be efficacious. Moreover, a significant number of lymphedema patients are obese or overweight and, therefore, would not be readily able to utilize off-the-shelf garments. Thus, accurate measurement of patients for compression garment fitting, especially in relation to the generation of precisely-fitted custom compression fit garments, remains a need.

Surgeons and oncologists were previously taught that treatment could wait until patients reported symptoms or swelling became visible. There is now a body of evidence demonstrating that waiting to treat BCRL when it becomes visible and symptomatic may not be optimal. Just as with breast cancer itself, lymphedema can be detected at early, even a subclinical, latent stage amenable to treatment and that may reverse the progression to chronic, irreversible lymphedema. In this regard, a 2008 NIH study revealed early diagnosis of lymphedema in breast cancer patients (“stage 0”) associated with an early intervention, fitted with a compression sleeve and gauntlet for one month, led to a return to preoperative baseline status. In a five-year follow up, these patients remained at their preoperative baseline, suggesting preclinical detection of lymphedema can halt, if not reverse, its progression.

The diagnosis or early detection of lymphedema can be difficult. The first signs may be subjective observations such as “my arm feels heavy” or “I have difficulty these days getting rings on and off my fingers”. These may be symptomatic of early stage of lymphedema where accumulation of lymph fluid is mild and not detectable by any difference in arm volume or circumference. As lymphedema develops further, definitive diagnosis is commonly based upon an objective measurement of differences between the affected or at-risk limb and the opposite unaffected limb, e.g. in volume or circumference. In most cases, while the common practice patterns that are currently employed, a clinical diagnosis of lymphedema is made when the condition becomes visually evident and is usually classified as “mild” lymphedema, often defined as an initially reversible, about two-centimeter (cm) circumferential difference or about 200 mL limb volume difference between the affected and unaffected contralateral arms. Undiagnosed, or not treated effectively, lymphedema can progress into later stages of the condition resulting in a severe form of swelling called “elephantiasis.”

Currently, lymphedema detection, as well as compression garment fitting, is conducted primarily by use of a tape measure. While tape measurement techniques are widely available to a broad scope of medical providers, the technique generally suffers from poor accuracy. In short, tape measurement is not a very effective diagnostic tool because the current clinical threshold for a positive diagnosis of lymphedema is a 10% volume increase in a limb, while the tape measurement technique has been shown to exhibit as much as an about 8 to about 12% inaccuracy due to inter and intra-operator variability. Such variability gives rise to a need for methods of detection that demonstrate improvements in both accuracy and precision.

Water displacement can provide very accurate volumetric measurements, if performed correctly, but this technique is rarely implemented in a clinical setting due to challenges with implementation and hygiene concerns. In short, few clinical settings are able to install and maintain a water tank of the size needed to accurately generate body part volume measurements or geometries needed to diagnose and treat lymphedema. Volumetric measurement, when available, can allow for diagnosis of the onset of lymphedema in a patient, however, this measurement technique cannot be used to size and fit compression garments used to treat patients diagnosed with lymphedema.

The only other current method available to generate accurate volumetric measurements is a high-end 3D scanner, Perometer®. However, the price of this device, believed to be about $30K, prevented widespread adoption of this device in the US. Accordingly, as of the time of filing of the instant application, the Perometer is no longer sold in the US.

In 2012, the American Cancer Society recommended that patients at risk for lymphedema should be monitored regularly to ensure that symptoms are caught at an early stage so as to prevent the condition from becoming chronic. Increased patient access to methods of detection is therefore paramount, even while patient access to clinicians can be restricted due to patient distance from clinicians, lack of necessary equipment in clinics and hospitals, and lack of available medical providers with training to use existing diagnosis techniques.

Recently, lymphedema diagnosis using readily available imaging devices has been proposed. US Patent Publication No. 2015/0302594 discloses the use of a structured light imaging device to generate a body part measurement that is stated to be usable to diagnose lymphedema. US Patent Publication No. 2015/0216477 discloses, among other things, a method of using various types of imaging devices to detect body part volume measurements or geometries that can be used to detect lymphedema. Neither of these references, each of which are incorporated herein by reference, discloses methods of imaging a patient in need of monitoring for the possible onset of lymphedema by implementing a simple-to-use imaging technique that, for example, can be operated by the patient herself in home or in an extra-clinical setting, such as a physical therapist's office. The present invention provides this and other benefits.

### SUMMARY OF THE INVENTION

The present invention comprises methods for detecting, monitoring and treating lymphedema by using certain imaging devices, in particular, a time of flight imaging (“ToF”) device, to generate anatomical information and measurements from which body part volume measurements or geometries can be derived. Patients in need of monitoring include those who have experienced disruptions to their lymph systems, such as in cancer treatments. The methods herein enable simplified body image acquisition and processing, thereby enhancing patient access to lymphedema detection, monitoring and treatment. Yet further, the present invention comprises methods of fitting compression garments for treatment of lymphedema by using the anatomical information generated according to the described methods. Body parts that can be assessed in accordance with the methodologies herein include one or more patient arms or legs, trunk, neck, or any other body feature that might be susceptible to the exhibiting lymphedema.

In some aspects, the invention herein comprises generating a plurality of images of four or fewer sides of the patient, that is, front side, left side, back side and right side. These plurality of images of each of the patient sides can be processed using at least a depth map generation step and a body part identification step. In some aspects, the image processing steps can also include one or more of a joint identification step; a first body part alignment step; a second body part alignment step; and a 3D reconstruction step. The number of sides to be imaged, and the number of image processing steps is dependent, in part, on the body part being analyzed as discussed further herein.

The methods of the present invention are particularly suitable for allowing a patient to be imaged in extra-clinical settings, such as the patient's home or other setting where medical imaging equipment and personnel are not generally available. Accordingly, the methods of the present invention enhance the ability to detect, monitor and treat a patient for symptoms and indications of lymphedema, as well as to fit compression garments to the patient. Patient range of motion assessment is also possible with the methods of the present invention.

Additional advantages of the invention will be set forth in part in the description that follows, and in part will be apparent from the description, or may be learned by practice of the invention. The advantages of the invention will be realized and attained by means of the elements and combination particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIGS. 1a and 1b present front and back views of a patient position for imaging according to one aspect of the invention.

FIGS. 2a and 2b present an imaging device and patient position for imaging according to an aspect of the invention.

FIG. 3 sets out exemplary process steps for obtaining an arm volume measurement according to an aspect of the invention.

FIG. 4 illustrates front, back and collective joint maps according to an aspect of the invention.

FIG. 5 illustrates shoulder identification from the joint map of FIG. 4 according to an aspect of the invention.

FIG. 6 illustrates arm alignment from the joint map of FIG. 4 according to an aspect of the invention.

FIG. 7 illustrates isolation of an arm from a patient depth map after arm alignment according to an aspect of the invention.

FIGS. 8a and 8b illustrate isolation of upper and lower arm portions, respectively, from a patient depth map according to an aspect of the invention.

FIG. 9 illustrates an aligned body image according to an aspect of the invention.

FIGS. 10, 11a, 11b, 11c, 11d and 12 illustrate 3D reconstruction steps according to an aspect of the invention.

FIG. 13 illustrates a 3D mesh from which anatomical measurements can be derived according to an aspect of the invention.

FIG. 14 illustrates 3D reconstructed arm segments from which patient arm volumes can be derived according to an aspect of the invention.

### DETAILED DESCRIPTION OF THE INVENTION

Many aspects of the disclosure can be better understood with baseline to the Figures presented herewith. The Figures are intended to illustrate the various features of the present disclosure. Moreover, like references in the drawings designate corresponding parts among the several views. While several implementations may be described in connection with the included drawings, there is no intent to limit the disclosure to the implementations disclosed herein. To the contrary, the intent is to cover all alternatives, modifications, and equivalents.

The term “substantially” is meant to permit deviations from the descriptive term that do not negatively impact the intended purpose. All descriptive terms used herein are implicitly understood to be modified by the word “substantially,” even if the descriptive term is not explicitly modified by the word “substantially.”

The term “lymphedema” may include either primary or secondary lymphedema, the latter of which might also be term “acquired” lymphedema. As would be recognized, primary lymphedema is caused by abnormal development of the lymph system. Symptoms can be present at birth, or may appear later in life. Secondary lymphedema is caused by damage to the lymphatic system. The lymphatic system may be disrupted, damaged or blocked by infection, injury, cancer, removal of lymph nodes, radiation to the affected area or scar tissue from radiation therapy or surgery. In significant aspects, the present invention provides methods of detection of lymphedema that occurs as a result of removal or damage to lymph nodes that occurs after treatment of a patient for breast cancer.

As used herein, the phrase “detecting lymphedema” or “diagnosing lymphedema” means detecting or diagnosing the existence of lymphedema, as well as the onset of lymphedema, early stage lymphedema and the progression of lymphedema over time. Thus, methods for detecting lymphedema can also be used to monitor the progression of lymphedema over time and treat/manage lymphedema in an individual.

In broad constructs, the methods of the invention relate to determining a change over time of the volume or geometry of one or more of a patient's body parts, where the change can be determined by comparing body part volume measurements or geometries taken at one or more baseline measurements with body part volume measurements or geometries taken at subsequent times. Such measurements are derived from anatomical information and measurements generated according to the image acquisition and processing methodologies herein.

A patient may be symptomatic of lymphedema, and lymphedema may therefore be diagnosed or detected, when the baseline body part volume measurements or geometry measurement is at least about 10% or about 7.5% or about 5% or 3% or about 1% or about any value less than about 10% different from a subsequent body part volume measurements or geometry measurement of a corresponding limb. Comparison is typically conducted in a corresponding limb that is a contralateral limb, for example patient left arm vs. right arm or left leg vs. right leg, but, in some scenarios, the corresponding limb is the same limb. In some aspects, a body part volume measurements or geometry measurement is indicative of the appearance of lymphedema in a patient when the subsequent body part volume measurements or geometry measurement shows a difference of about 1% or about 3% or about 5% greater than the baseline measurement, where such body part volume measurements or geometries can be adjusted for patient weight differences between measurement events, if any. The body part volume measurements or geometries can be taken of all or part of one or both of the patient's arm, just the lower arm or just the upper arm. The patient's body part volume measurements or geometries can also be taken of all or part of one or both of the patient's legs. Still further, body part volume measurements or geometries can be taken of the patient's arms, legs, truck, neck or genitals.

To generate more accurate detection or diagnosis of the occurrence of lymphedema in a patient in need of detection or diagnosis, the baseline body part volume measurements or geometry measurement(s) should be taken as close as practicable to the time when the patient undergoes a procedure that makes her susceptible to becoming symptomatic of lymphedema resulting from a disruption of the patient's lymphatic system. Subsequent body part volume measurements or geometries can be taken at intervals concordant with the medical practitioner's standard of care. In some aspects, the subsequent tests can be conducted on a daily, weekly or monthly basis, where such subsequent measurements are monitored to determine whether, and to what extent (if any), the patient's body part volume measurements or geometry measurement is increasing over the course of the monitoring as compared to the baseline body part volume measurements or geometry measurement.

In significant aspects, the methods of the present invention are appropriate for generating body part volume measurements or geometries of a patient's body areas to, for example, prevent, diagnose and/or treat lymphedema in a patient in need of treatment. The methodology herein is suitable for addressing potential or actual lymphedema of one or more of the upper extremity, lower extremity, head or neck, head or genital areas.

In further significant aspects, the methods herein can enhance patient access to regular body part volume measurements or geometry measurement monitoring for the appearance of lymphedema symptoms because the imaging process is simple-to-use in that the imaging steps can suitably be operated by a patient or non-medical personnel. As such, the methodology herein can allow more frequent patient monitoring than is normally possible with existing lymphedema detection and monitoring methodologies. In some aspects, the patient imaging step can occur in a location remote from the patient's medical team. For example, the patient imaging step can be conducted in the patient's home or other non-clinical setting.

Broadly, the patient scan can be performed using a ToF imaging device by taking a “front half” scan (patient positioned facing forwards), a “back half” scan (patient positioned facing backwards), and a side view scan from each of the right and left sides (patient positioned facing right and left, respectively). In some scenarios, as discussed further herein, fewer than four scans can be used, such as when the patient's trunk or neck is being examined for lymphedema symptoms or indications. These about four or fewer patient positionings can be used to assemble a three-dimensional reconstruction of the patient so as to generate anatomical information of the pertinent patient body part(s) from which body part volume measurements or geometries can be derived. The scans generated in each of the four or fewer patient positionings, each, independently, comprise a plurality of images of the patient. In particular, the imaging step generates a plurality of front view patient images, left side view patient images, back side view patient images, and right side view patient images.

In generating the plurality of images in accordance with the present invention, the patient is likely to shift her position when moving among the imaging positions. Joint mapping and various alignment steps, as discussed in more detail below, can be used to ensure the imaged positions of the patient are consistent in the front scan and the back scans, which can allow more accurate anatomical information and, thus, more accurate body part volume measurements or geometries to be derived from the plurality of patient images generated from the imaging device.

In a first aspect, the invention comprises generating baseline patient anatomical information of one or more patient body areas with which to compare subsequent patient anatomical information. In some aspects, the baseline anatomical information can comprise measuring with the imaging devices as discussed elsewhere herein one or more of the patient's extremities, that is, one or more arms or legs. Still further, the baseline anatomical information can be generated from one or more body portions of the patient. The baseline anatomical information can be generated prior to or shortly after a risk inducing surgical procedure, such as a mastectomy or other lymph node-damaging procedure, or at a time thereafter. If baseline measurements are generated after the risk inducing procedure, the baseline measurement should be generated substantially close in time to that procedure so as to better enable accurate diagnosis of a difference in body part volume measurements or geometry that can be correlated with a change that could be attributed to the patient being symptomatic of the appearance of lymphedema.

The first time such body part volume measurements or geometries are generated of a patient, such first body part volume measurements or geometry measurements can serve as a baseline body part volume measurements or geometry measurements. Subsequent body part volume measurements or geometries generated in subsequent imaging events can then serve as a diagnostic tool to determine whether a patient is symptomatic of lymphedema. After the baseline measurements are generated, the patient can be periodically monitored with additional imaging, where such subsequent measurements are generated according to the methodologies discussed herein.

In addition to enhancing diagnosis of lymphedema, the methodology of the present invention can also be useful for regular monitoring of a patient who has been diagnosed with lymphedema. If one or more body part volume measurements or geometries change between measurements from the baseline volume measurement of the same body part, the patient may obtain a diagnosis of lymphedema by a medical provider who reviews her medical records. Such diagnosis can also occur automatically in accordance with a rules engine that will generate a diagnosis of lymphedema if a subject body part volume measurement or geometry has increased over the baseline measurement. Software in which such a rules engine is incorporated can notify the medical staff that the subject patient may be symptomatic of lymphedema. Such regular monitoring can be expected to both improve treatment outcomes, as well as to reduce medical costs for treating the chronic condition of lymphedema. Regular testing by a patient at risk for developing lymphedema is greatly enhanced by the simplified imaging acquisition methodology herein, and can allow a patient to be closely monitored in a telemedicine or an environment where licensed medical providers may be scarce.

In some aspects, at least one subsequent body part volume measurement or geometry measurement is generated to provide one or more subsequent body part volume measurements or geometries. In accordance with the present invention, such subsequent body part volume measurements or geometries can be generated about one or about two or about five or about ten or more additional times, from about week to week to about month to month or about year to year, or any other intervals as directed according to a medical protocol applied to the subject patient. The associated software can be configured to notify the patient via email, text etc. that she needs to perform a body imaging event. To more accurately identify the early stages of lymphedema occurrence, which as discussed previously, is much more treatable if detected in an early stage, subsequent measurement events should enable to generate anatomical information from which small changes small changes in the patient's body part volume measurements or geometry over time. Moreover, the technique used must be reproducible with regard to enabling the patient's body part volume measurements or geometry to be tracked closely over a time period. In this regard, the technique must not allow significant variability within the same patient from images and, thus body part volume measurements or geometries, generated in various imaging events. In short, the measurement technique must be both accurate and precise. Such accuracy and precision should also be possible even though the imaging step might be conducted by a person without medical and/or imaging training.

Such accuracy and precision has been found by the inventors herein to be available from use of ToF imaging techniques used in conjunction with the patient imaging positioning of the present invention. In significant aspects, the detection method of the present invention utilizes ToF imaging, as such term and the attendant technique is discussed hereinbelow. Yet further, the detection method of the present invention consists essentially of ToF methodology.

In conjunction with the detection of lymphedema, and the attendant need for accuracy and precision in longitudinal measurements, the inventors have found that there is a significant difference in measurement accuracy within and between measurements between structured light and ToF devices. As such, unlike the structured light methodology disclosed in US Patent Publication No. 2015/0302594, the disclosure of which was previously incorporated by reference, structured light is not utilized in the detection method herein.

In a first aspect, structured light imaging differs from ToF imaging, in part, because each obtains depth detection differently. As would be recognized, the structured light approach is an active stereovision technique. Thus, an image taken with a ToF device is different in kind from an image taken with a structured light device. For example, structured light generates direct measurements only at scene points that are suitably illuminated by a projected light pattern. Depth values at non-illuminated points must therefore be derived via interpolation or surface fitting. To obtain dense depth information, such as is necessary to generate accurate body part volume measurements or geometry data from a patient to obtain accurate body part volume measurements or geometries as required herein, interpolation between these pixels is essential. As would be recognized, interpolation and surface fitting will greatly increase the processing needs and algorithm sophistication required to obtain accurate measurement data. In contrast, the inventors herein have found that, due to their high lateral resolution capabilities, ToF imaging devices can provide dense depth information at constant resolution and high frame rates without the need of interpolation. The inventors herein have determined that this capability of ToF imaging devices can provide higher depth resolution and more consistency in measurement data as compared to measurement data derived from structured light. Such benefits have been found to provide, in part, the ability to generate patient images using, for example, a plurality of patient images generated from front, back and left and right side positioning of the patient, as opposed to the full patient body imaging as required by prior art methods.

In a further aspect, while both structured light and ToF are able to provide measurement data in real time, as applied to lymphedema detection and the attendant measurement data, the inventors herein have found that the accuracy of measurements generated from structured light usually drops when trying to generate measurements in real time. This results in low accuracy depth measurements associated with movement. This difference is significant in relation to the invention herein: given that human subjects will always shift their positioning of arms, legs etc. somewhat during imaging of the different patient sides, especially those who are weak from old-age or when recovering from medical procedures, use of ToF imaging provides the ability to maintain high resolution of the patient during real-time imaging, thereby providing a higher resolution depth map than is obtainable from structured light. As a result, in significant aspects, the present invention allows body part volume measurements or geometries to be obtained substantially in real time.

Yet further, unlike with structured light systems, the present invention substantially does not require calibration in order to map the observed light pattern to 3D point values. Besides calibrating the imaging device itself, the relative geometry of the light rays with respect to the imaging device has to be known to obtain measurements from a structured light imaging device. Accordingly, the projection unit has to be mounted very accurately and is also highly application dependent, which leads to higher costs. In contrast, ToF imaging devices are monocular all-solid-state imaging devices, hence calibration is not required and hardware setup does not influence the accuracy. Accordingly, in a significant aspect, the invention substantially does not require calibration prior to generating images from which body part volume measurements or geometry data is derived.

In a further aspect, ToF imaging devices have been found to exhibit reduced sensitivity as compared to structured light to ambient light and other background effects that can reduce the quality of images from which anatomical information/measurements and, therefore, body part volume measurements or geometry measurement data will be derived. Accordingly, body part volume measurements or geometry measurement data generated from ToF imaging devices provides more consistent measurement data when measurements are generated longitudinally. Such lower sensitivity enhances the ability of the imaging step to be conducted in an extra-clinical setting, such as a patient's home or a physical therapist's office.

Imaging devices suitable for use herein comprise ToF devices that are commercially available. An exemplary ToF device that is well-suited for use with the methodology herein is the Microsoft® Kinect® 2nd Generation device. This 2nd Generation Kinect is to be distinguished from the 1^{st} Generation Kinect, as the latter uses a structured light technology. Other ToF imaging devices that are now or that may later be in the market can also suitably be used in the present invention.

Imaging in accordance with the present invention comprises a ToF imaging apparatus. The ToF imaging device is configured to measure depth, but potentially including but not limited to: at least one RGB imaging device; at least one depth (IR) imaging device; and one or more accelerometers to track and capture the movement of the imaging device in space. Yet further: ToF imaging devices for use in the present invention comprise the following features:
A) an illumination unit, where the illumination unit can comprise infrared light to minimize any effects on the patient;
B) a lens/optics to gather the reflected light and onto the image sensor (focal plane array);
C) an image sensor: measures the time the light has taken to travel from the illumination unit to the object and back to the focal plane array;
D) driver electronics: both the illumination unit and the image sensor can be controlled by high speed signals and synchronized;
E) computation/interface: The distance can be calculated directly in the imaging device and the imaging device then provides the image information over a network connection that can be wired.

The previously disclosed lymphedema detection method in US Patent Publication No. 2015/0216477, the disclosure of which was previously incorporated herein, does not disclose any patient positioning information or image acquisition instructions. The '477 Publication, while generally disclosing detection of lymphedema using ToF imaging devices, provides no information on how a patient should be positioned to generate suitable images. Because proper positioning of the patient is essential to generate images from which accurate body part volume measurements or geometries can be obtained, the '477 Publication does not enable generation of accurate anatomical information and, therefore, of accurate body part volume measurements or geometries from a patient. Additionally, the inventors herein believe that the image acquisition step of the '477 Publication requires the imaging device to travel fully around the patient and/or the patient to be rotated in place while remaining stationary, such as with the aid of a mechanical turntable or the like. Such full rotational engagement of either or both the imaging device and the patient is indicated by use of the Kinect2 developer kit (SDK 2.0 by Microsoft) referenced therein. In contrast, the image acquisition step of the present invention substantially does not require the imaging device to travel around the patient or the patient to fully rotate around the imaging device while the patient remains substantially stationary in order to generate a plurality of images from which patient anatomical information can be derived and from which one or more body part volume measurements or geometries can be obtained.

Instead, the inventors herein have determined that it is possible to obtain one or more body part volume measurements or geometries maintaining the ToF imaging device substantially or fully stationary, such as being placed on a table or mounted on a stand, and having the patient position herself in about four or fewer positions (e.g., front, left side, back, and right side) whereby a plurality of images suitable from which to generate body part volume measurements or geometries can be derived. Such plurality of images can be used to derive body part volume measurements or geometries that can then be used to detect lymphedema as described elsewhere herein.

As discussed in more detail below, the ToF imaging device is suitably placed on a table, a tripod or otherwise within about 3 to about 8 feet away from the patient who will be imaged. The imaging device height is placed about knee high to about shoulder high relative to the patient being imaged. The placement of the imaging device is dependent, in large part, on the body part being measured. In this regard, the imaging device placement when only the arms are being measured can be placed so that substantially only the upper body is visible in the images. Similarly, if only the lower body is being measured, the imaging device can be placed so that substantially only the patient area below the waist is visible in the images. However, because significant information can be generated from a substantially full patient body imaging, it is often useful to place the imaging device so that substantially all of the patient's body is visible in the plurality of images.

The images can be acquired quickly to reduce the tendency of the patient to become fatigued and to reduce the propensity of the patient to inadvertently alter the positioning of the relevant body parts during image acquisition in the about four or fewer positions. In some aspects, the images are taken at about 10 or about 3 or about 5 or about 10 or about 20 or about 30 or about 40 frames per second (fps). In some aspects, the image acquisition for each of patient's sides can occur in less than about 5 or less than about 3 or in about 1 seconds, where the time of image acquisition depends largely on the speed of the imaging device, the number of images acquired of each side, and how quickly patient moves (or is assisted in movement by another person) through the image acquisition step.

With regard to positioning of the arms relative to the body, the arm angle should be large enough such that the patient's armpits can be identified from the plurality of images. The armpits can serve as an anatomical reference point for the identification of the shoulder in the images. Patients who are larger will require a larger angle to fully separate the upper arm from the torso, whereas thinner women will achieve arm/torso separation at a smaller angle. Similarly with the legs, the patient should spread her feet wide enough to create a separation between thighs to generate a “thigh gap.” This serves as an anatomical reference point to identify the upper bound of the leg for segmentation. Some women cannot feasibly spread their feet wide enough to create a true separation, in which case they can be provided with instructions to spread their feet as wide as is comfortable, and the software can be configured to measure and record that position in order to instruct them to assume a similar position for future scans.

Proper positioning of the patient for imaging can be enabled by providing the patient with instructions directing her to place her body in each of the about four or fewer body positions (e.g., front, back and each of the left and right sides or some combination thereof) used to generate the plurality of images from which body part volume or geometries are derived. Instructions can also be provided directing the patient in proper arm and body placement to generate this plurality of images. Written collateral can be provided to the patient in some aspects. Still further, the software instructions associated with the methods herein can include instructions on patient positioning, including by incorporating diagrams or photographs to assist the patient in positioning. Still further, software associated with the methods herein can be configured to assess the positioning of the patient in real time, such as by determining whether the patient is facing the right direction and/or positioning her legs correctly. If the positioning is correct for the position being imaged at that time, the imaging can start. If the positioning is not correct, the software can be configured to instruct the patient to move to the appropriate position, with further position confirmation steps occurring therefrom. For example, software associated with the imaging step can further include voice prompting for the patient if the imaging device senses that the patient is not properly positioned for appropriate scanning. For example, a voice command can say “raise your right arm slightly,” or “hold still for 5 seconds.”

In further aspects, the imaging device from which body part volume measurements or geometries are generated can be utilized with a placement device that can provide direction for the user to generate accurate measurements. While the imaging methodology of the present invention has been found to provide substantially accurate lymphedema detection, in some aspects, it can be beneficial to provide guidance for the patient or her assistant during the image capture process. For example, a template where a skeleton or outline of the patient can be affixable or projectable onto a wall, where the template provides instructions for the patient on the position in which she is required to stand in order to generate the plurality of images from which the body part volume measurements or geometries can be derived. For example, the template can first incorporate a front projection of a patient outline with her arms at about 30 or about 45 degrees or about 60 or about 90 degrees, then switch to left side projection, back side projection and right side projection. The projection can be provided by light or the like projectable from the ToF imaging device used to generate the plurality of images.

As noted, the plurality of images from which lymphedema can be detected, monitored or treated can be generated from four or fewer patient positionings. In this regard, the number and type of image processing steps used in a particular scenario will depend, in part, on the body part being analyzed and the level of detail needed for that scenario.

For example, when full arm (or leg) volume calculations are appropriate, the image processing steps can include the step of: depth map generation, joint identification, first body part alignment, body part identification, second body part alignment and 3D reconstruction. Such additional steps can enhance the detail obtainable from the plurality of images of the patient's body. When such enhanced detail is desirable, a plurality of images of each of the patient's four sides (e.g., front side, left side, back side and right side) will be generated as described elsewhere herein.

In a further example, measurements can be based only on one scan, such as where substantial information about a local body part of interest is obtainable from only a single scan, such as where lymphedema would be symptomatic in the patient's breast/trunk. In such a case, only a front side patient scan would generally be indicated. Another example could be head/neck lymphedema that is localized on one side of the body. In one scan measurement, the depth map, body part identification and geometry measurement would be generated. A joint identification step may optionally be conducted, such as to enhance the ability to identify a relevant body part. In this regard, body part identification could be based on joints or it could be based upon other anatomical landmarks such as the mouth or chin in head/neck lymphedema or armpit, clavicle, and sternum for breast/trunk lymphedema.

In a further example, two body scans could be generated to provide body geometry measurement. In such a scenario, less precise image alignment would generally be required. Examples would encompass large volume head/neck or trunk lymphedema indications where it would be appropriate to conduct at least two, but less than four scans to generate full measurements of the relevant body parts. Steps included in this scenario would be depth map generation, first body part alignment, 3D reconstruction, body part identification and geometry measurement.

The plurality of patient front side view, left side view, back side view and right side view images can be processed according to methodologies suitable for analyzing images as is known. The image processing steps can include at least a depth map generation step and a body part identification step. In some aspects, the image processing steps can also include one or more of a joint identification step; a first body part alignment step; a second body part alignment step; and a 3D reconstruction step. These steps are described in more detail in regards to generating an arm volume measurements hereinbelow.

Still further, the present invention provides an improved method for generating the volume or geometry of a patient's body and/or extremities for the actual or potential presentation of lymphedema. As such, accurate measurements can be generated in virtually any location that the patient might be located using the image generation and processing methodologies disclosed elsewhere herein. Moreover, the relative simplicity of generating the plurality of images from which the body part volume measurements or geometry can be derived makes it possible for patients to take their own images in convenient locations. The increased simplicity provides a previously unrealized ability to enable regular monitoring of patient body part volume measurements or geometry in, for example, a home setting or in other extra-clinical settings, thereby greatly enhancing the ability to detect and monitor, and therefore proactively treat, lymphedema.

In significant aspects, the methodology herein allows a patient in need of monitoring for the appearance of lymphedema to be repeatedly and continuously monitored over time to assess whether treatment with compression garments is indicated. For example, disease progression (or non-progression) can be monitored in a patient to determine whether the patient may be pre-symptomatic of lymphedema or need initial compression garment fitting or adjustment of compression garment fitting. In some aspects, the patient in need of monitoring can be measured for anatomical measurement changes from day to day, week to week, month to month or year to year. Using the ToF imaging methodology herein, such measurements will be accurate over time, even when the technician taking the measurements changes between one or more patient visits and/or the location where the measurements are taken changes.

In some aspects, the present invention can account for weight fluctuations by a patient between and among measurement events. In this regard, the patient's weight can be recorded along at about the same time of each imaging event, and this weight associated with that imaging event. If the patient's weight fluctuates up or down between and among measurement events, a size differential value can be incorporated into the body part measurements as is known. One example of a size differential adjustment is disclosed in the '477 Publication, previously incorporated by reference. In a further aspect, non-lymphedema associated weight gain can also be assessed could be determined by the imaging device itself by observation of the patient's body at the torso, abdomen, or global volume changes. For example, software associated with the imaging device can be configured to analyze whether a size differential in the patient's arm is consistent with a size differential visible in the rest of the patient's body, where both size differences are derivable from the images obtained of the patient's body in accordance with the methodology set out herein.

In further aspects, the present invention provides improved methods of fitting compression garments for the prevention of or treatment of lymphedema using compression garments. Presently, compression garments are sized using a trained lymphedema therapist who takes serial tape measurements along the limb(s) in question. These measurements are then sent to custom garment manufacturers who create the garments to the specifications. However, these garments will largely only be effective if the measurements are accurate, and there is a significant amount of error in the tape measurements. Using the accurate and precise anatomical measurements generated by the methodologies of the present invention, inter and intra-operator variability can be substantially reduced, which can improve the fit of the garments and the outcomes of the patients. The body part volume measurements or geometries can be used to provide a plurality of serial circumference measurements generated along the body part of interest. The circumference measurements can be taken at very small increments, for example, as low as millimeter increments, if necessary, to provide a high-resolution rendering of the limb for garment sizing. Moreover, the ease of ongoing monitoring provided with the present invention allows the need for adjustments in compression garments to be determined and, thereby, more readily changed without the need for the patient to visit a medical provider. Methods of fitting compression garments, albeit using less effective prior art methods, are further described in U.S. Pat. No. 6,415,525, the disclosure of which is incorporated herein in its entirety by this reference.

Another significant advantage of the anatomical information obtained in accordance with the present invention is the improved ability to generate true to life 3D reconstructions of patients. When therapists take a circumference measurements using tape measures, they must assume the shape of the patient's limb is a sphere, but this is almost never the case. Such approximations can limit the effectiveness of the garment that is fit to the patient for the treatment of lymphedema. With the measurement and fitting system of the present invention, a substantially accurate (that is, “true to life”) geometry of the limb can be recorded, especially for locally obscure geometries (as is often the case with lymphedema), which can markedly improve the fit of the garment and help to ensure that the compression is substantially uniform thereby ensuring a more efficacious lymphedema detection and treatment regime.

The anatomical information generated by the methods of the present invention can also be utilized to generate patient range of motion calculations. In this regard, range of motion can first be calculated by identifying as a region of interest one or more various anatomical landmarks using the steps outlined above. The relative location and angle of these regions of interest can then be calculated while the subject moves through a range of motion. As an example, the range of motion of an arm moving from a hanging rest next to the trunk of the body up towards a horizontal position with the arm out can be calculated by identifying a region of interest for the wrist, elbow, shoulder, and hip. As the subject raises her arm, the hip and shoulder locations should remain almost completely stationary. This is the baseline plane. The wrist and shoulder will move in an arc away from the body as the arm is raised. At the highest point, a line can be drawn connecting the wrist, elbow, and shoulder to determine the angle of the arm from the body. This angle can be tracked over time to measure change in range of motion. A similar technique can be used with the motion of any limb in any direction. Range of motion changes over time can also be generated.

In a further aspect, the present invention comprises methodology for patient tracking and data analysis. As noted, because accurate anatomical measurements can be generated by imaging conducted remotely, ongoing patient monitoring in a telemedicine environment can be facilitated. In this regard, the methodology described herein can be used in conjunction with software instructions to record, analyze and display substantially all data relating to the patient's body part volume measurements or geometries for inclusion in an electronic health records environment. Data associated with such patient tracking aspects can comprise one or more of: automatically linking patient data to associated clinicians; measurement, tracking, and analysis of relevant body part volume measurements or geometry(s); comparison of relevant body part volume measurements or geometry(s) to contralateral body part volume measurements or geometry(s) or known baselines; normalization of relevant body part volume measurements or geometry(s) to weight and body fat; diagnosis of altered body part volume measurements or geometry(s) over time; alerts sent to patient and clinician for significantly altered patient body part volume measurements or geometry(s); measurement, tracking, and analysis of a patient's range of motion; comparison of range of motion to contralateral range of motion or known baselines; diagnosis of significantly altered range of motion; diagnosis of non-improving range of motion; alert sent to patient and clinician regarding range of motion problem; measurement of body part volume measurements or geometry and assessment for proper rehabilitation garment; repeated measurement and assessment of ideal rehabilitation garment size; and alert sent to patient, clinician, and garment distributor for altered body part volume measurements or geometry and new ideal garment size.

Suitable software for recording, tracking and reporting the body part volume measurements or geometry measurement is currently available, for example telemedicine software products for patient monitoring, as would be recognized by those of ordinary skill in the art. The protocols, instructions for patients etc. useful in the present invention can be incorporated into existing software product frameworks, or customized software can be generated for use with the present invention.

As would be recognized, the ToF imaging device is appropriately operationally engaged with a computer network. This allows the imaging device to be operated by the patient herself or remotely operated, such as by a medical technician located in a different location from the patient. Such operational engagement also enables the patient images to be uploaded to a computer or into “the cloud” to allow processing of the images. Indeed, the processes described herein do not require that the imaging, image processing and diagnosis be in close proximity to each other. Each step can be completed remotely from the others since the patient imaging, image analysis, and diagnosis can be communicated over a network, that is, in the “cloud.” With the exception of the plurality of images generation step, which is intended to be generated by using a ToF imaging device in the presence of the patient with the imaging device positioned and operated in accordance to the disclosure herein, each of the steps of the process herein can be performed in a location remote from the patient.

In further aspects, the method of the present invention can further include additional hardware features that include one or more of: a scale to measure the patient's weight; a current delivery and sensing system to monitor the patient's body fat percentage, through the feet and/or the hands; sensors to monitor the patient's heart rate; sensors to measure the patient's blood pressure.

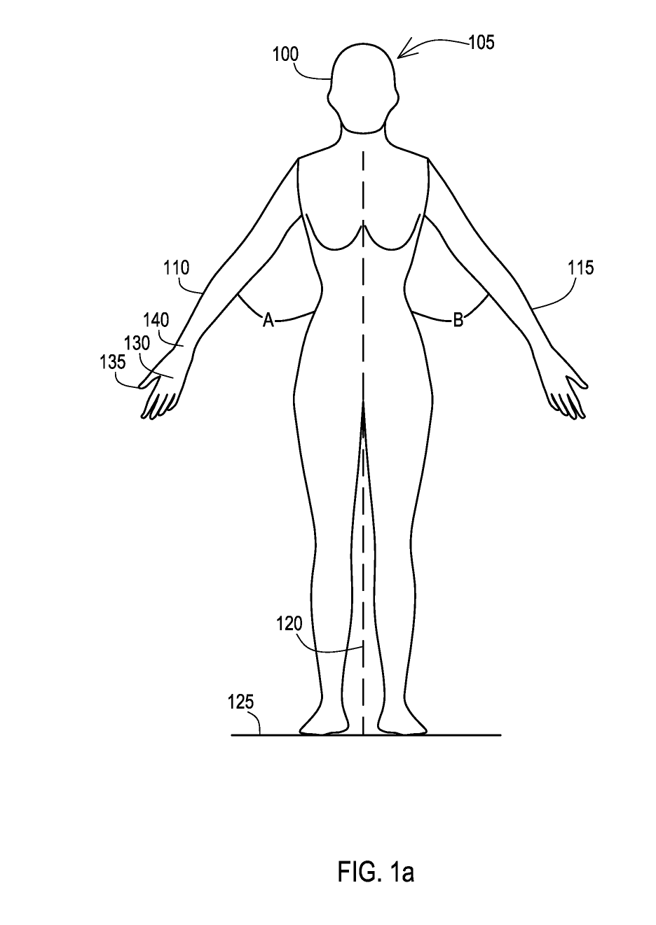

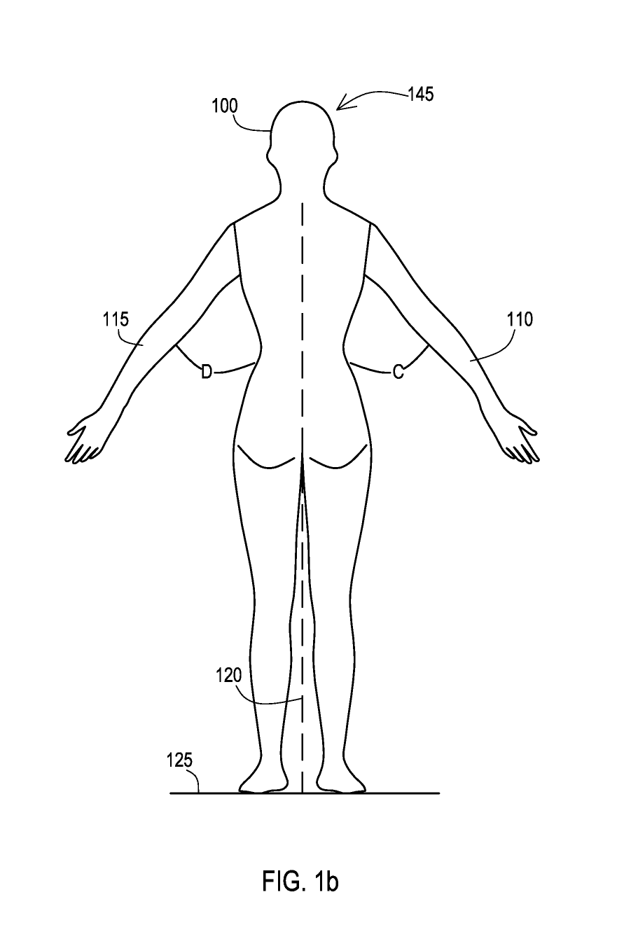

Referring now to the drawings, which present a process of generating an arm volume measurement, in FIG. 1a, patient 100 is present in front view 105. With regard to her right arm 110 and left arm 115, each are positioned for imaging at angles A and B, where these angles can be from about 10 to about 90 degrees and are measured relative to a vertical line 120 drawn perpendicular to a floor surface 125. Angles A and B will be approximately equal in the positioning instructions provided to the patient, but, in practice, they can vary both within a side imaging and especially among the various sides according to the ability of the patient to generate and maintain her position in accordance with instructions provided to her as described elsewhere herein. As shown in FIG. 1a, the imaging step can be performed with the patient's palm 130 facing generally forward and thumb 135 pointed in a generally upward position. In some aspects, this positioning helps to keep patient 100 position substantially consistent between scans and can enhance identification of an anatomical reference point for the wrist 140, which can, in turn, enhance the ability to align the various pluralities of side images, assist in identification of the arm parts and facilitate arm segmentation. In FIG. 1b, patient 100 is presented in back view position 145. Right arm 110 and left arm 115 will be positioned for imaging at angles C and D, respectively, where angles C and D are measured relative to the vertical line 120 drawn perpendicular to floor surface 125. Again, angles C and D can range between about 10 to about 90 degrees, and may be approximately the same or may differ somewhat.

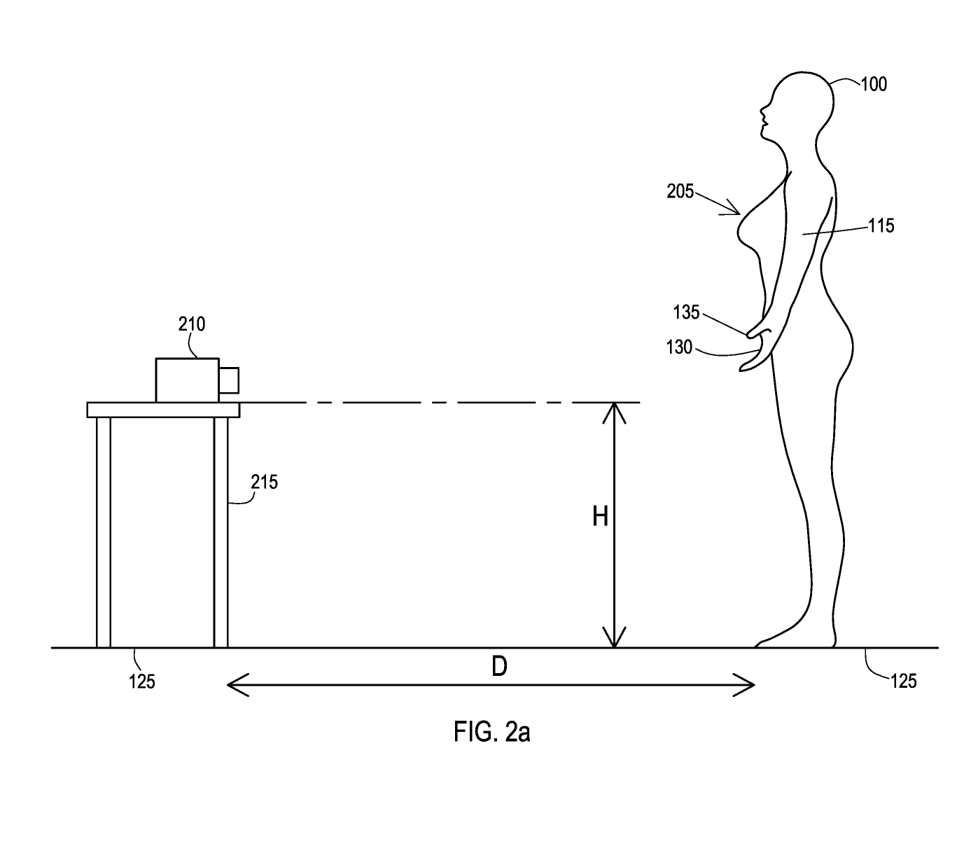

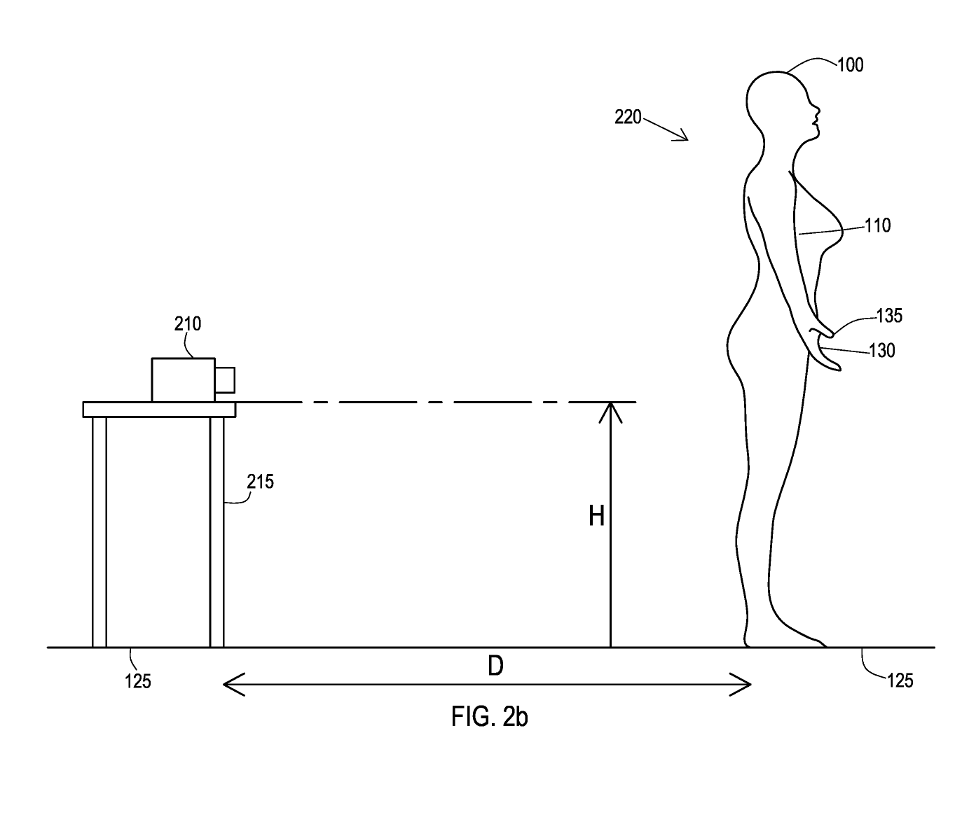

In FIG. 2a, front positioning 205 of patient 100 is shown where front images can be generated. In this regard, patient 100 is positioned a distance D from ToF imaging device 210. D can be from about 3 to about 8 feet from patient 100, with D determined, in part, by the height of the patient and the body parts being imaged. For example, if patient 100's right arm 110 (not shown) and left arm 115 are the body parts being imaged so as to generate anatomical measurements/information from which body part volume measurements or geometries can be derived, imaging device 210 need only be as far from patient 100 to allow suitable imaging of arms 110 (not shown) and 115. Distance D will also be influenced by the height H of the table 215. Proper positioning of patient 100 relative to imaging device 210 can be readily determined through routine experimentation by the patient 100, or through instructions provided. In FIG. 2b, rear positioning 220 of patient 100 to generate rear images of patient 100 is presented. While imaging device 210 is shown placed on table 215 in FIGS. 2a and 2b, it will be understood that a stand, tripod, chair or the like can be used to position imaging device 210.

When the patient's right arm 110 and left arm 115 are appropriately positioned, the ToF imaging device 210 is engaged, for example via software instructions, to take at least about 100 separate images of patient 100, when four patient views are imaged. In other aspects, at least about 60 or about 80 or about 100 or about 120 images of patient 100 can be obtained. An approximately equal number of images of each of patient 100's sides to generate the plurality of images of each side, or more or less can be taken. Patient 100 will be instructed to keep approximately the same position during rotation of her body for image acquisition on about four or fewer sides of her body, but there will be some variability in position as she moves through the imaging process, where such variability will require correction of alignment or the like, as discussed elsewhere herein.

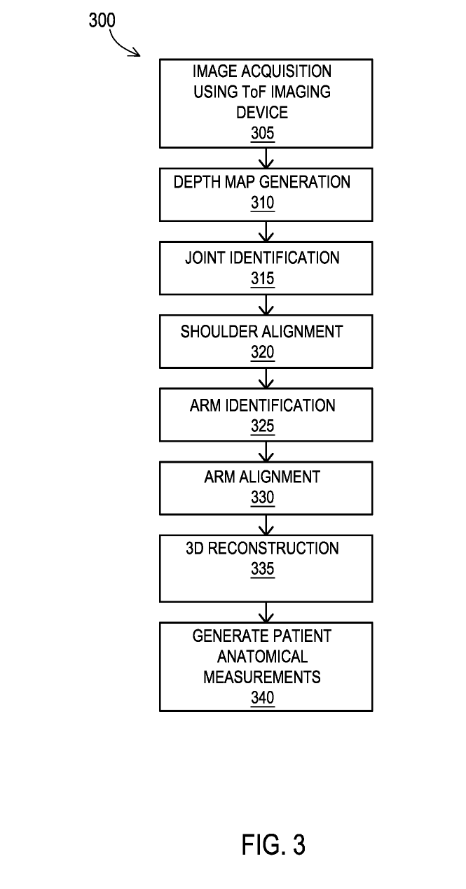

Referring to FIG. 3, the overall process of generating patient anatomical measurements is presented. After image acquisition step 305 as discussed above, a depth map generation step 310 is provided. As would be recognized, suitable depth maps can be generated by analyzing substantially all of the generated pixel and related information over all the frames taken in each patient positioning, that is, front side, left side, right side and back side. Information from such patient depth maps can be used to generate the body part volume measurements or geometries from which the lymphedema detection methods of the present invention are conducted. Alternatively, fewer than four sides can be imaged as discussed elsewhere.

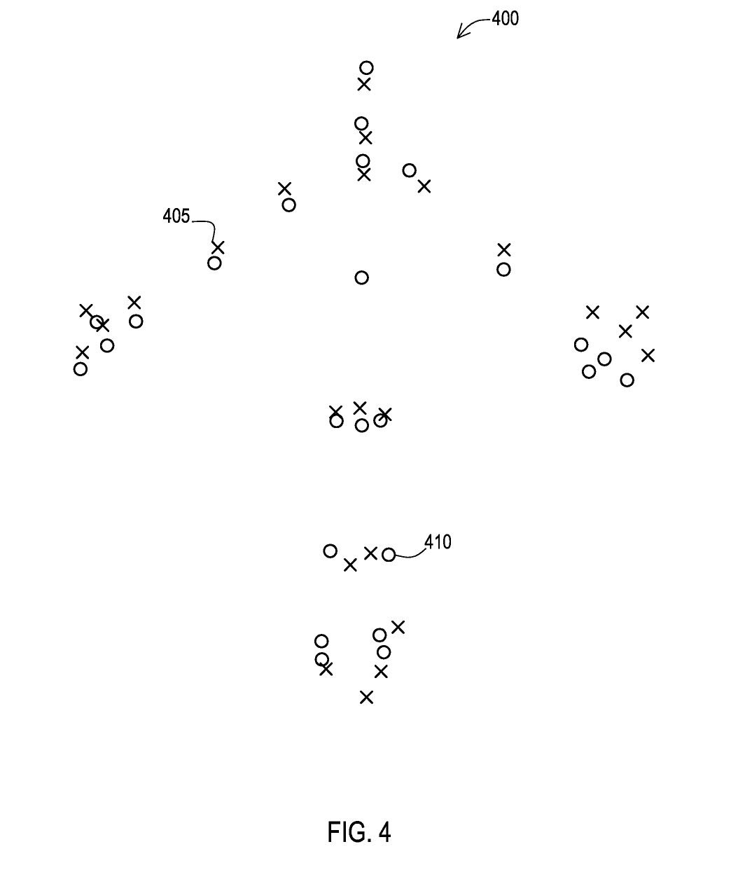

In the arm volume measurement discussed, the joints are identified in step 315 of FIG. 3. As shown in FIG. 4, collective joint map 400 is provided, where front joint map 405 and back joint map 410 are superimposed to create front and back joint map 400. Front joint map 405, which is represented by x's extracted from a depth map and back joint map 410 is represented by o's created, where such joint maps are each, independently, created from a plurality of patient images generated from a ToF imaging device as described elsewhere herein. Note that front joint map 405 is created from all identified joints as represented by x's in collective joint map 400. Similarly, back joint map 410 is created from all identified joints as represented by o's in collective joint map 400. Such joint locations are derived from depth map generation step 310, where the locations are identified by image processing. Joint maps and depth maps can be generated, in one aspect, by the native software available with Microsoft Kinect™ imaging device. Additional methods of obtaining depth and joint maps from imaging methods such as those used herein are disclosed in Cahyawijaya, Samuel, and Iping Supriana Suwardi. “Automatic Human Joint Detection Using Microsoft Kinect.” Conference Papers August 2014, available at https://www.researchgate.net/profile/Iping_Supriana2/publication/264983881_Automatic_Human_Joint_Detection_Using_Microsoft_Kinect/links/53fae50b0cf27c365cf051ef.pdf (retrieved Feb. 10, 2016), the disclosure of which is incorporated herein in its entirety by this reference.

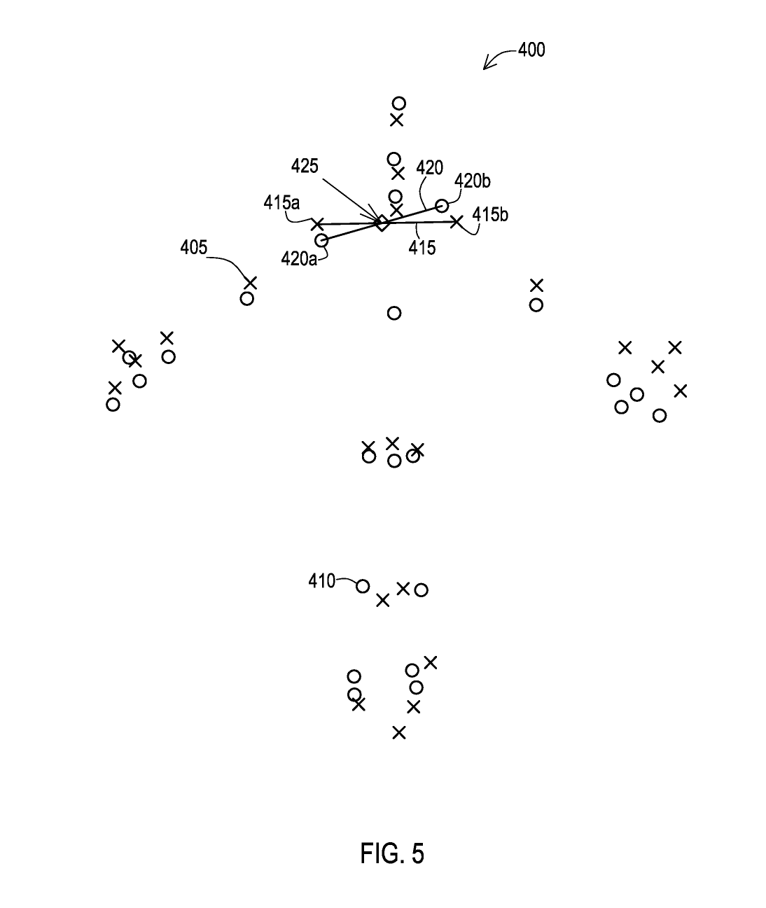

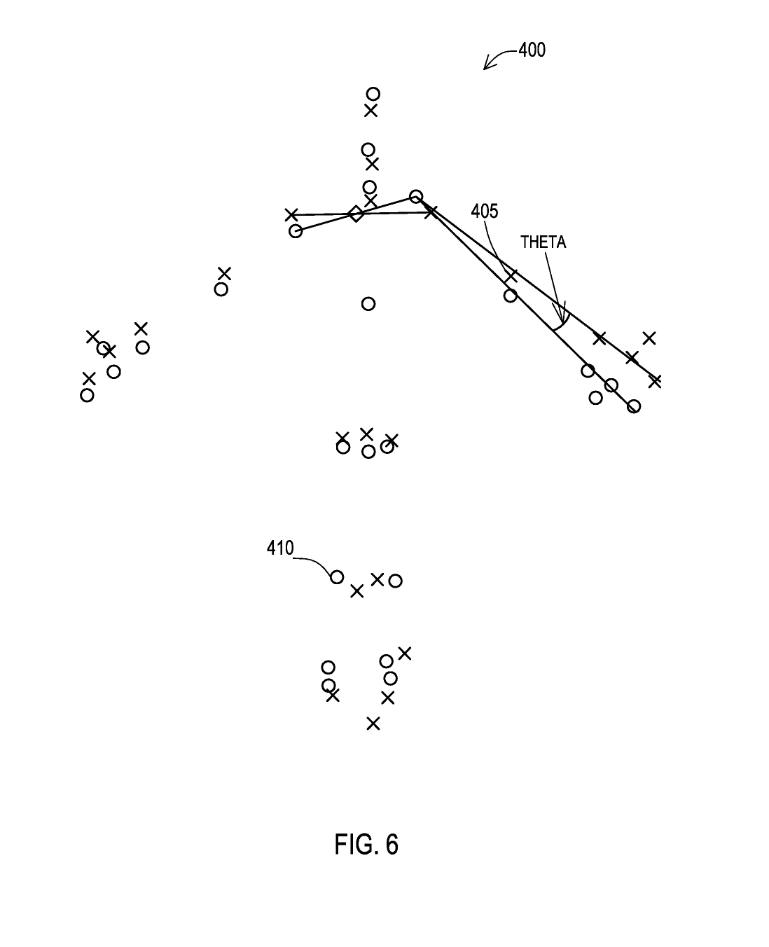

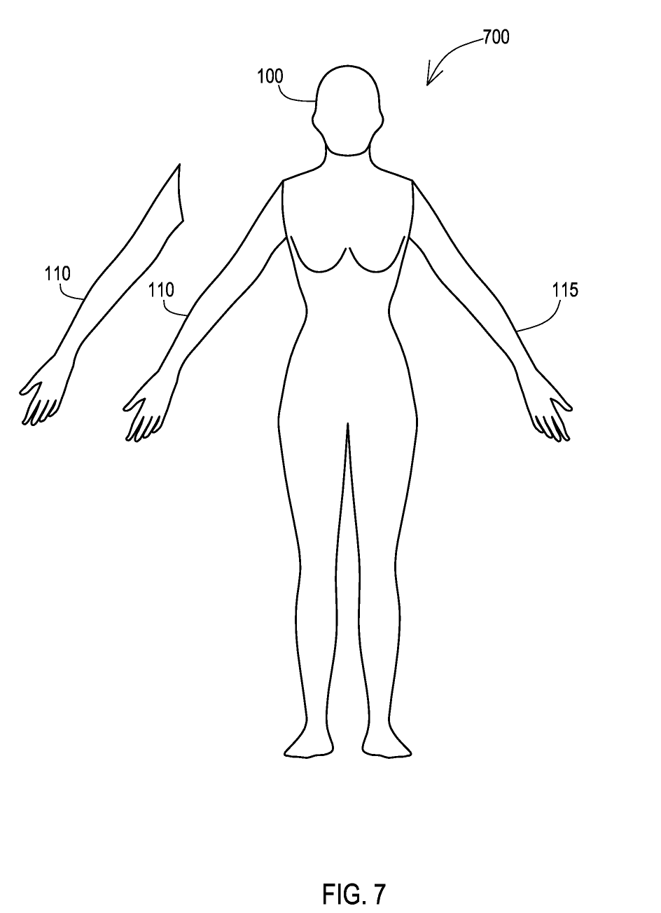

In step 320 of FIG. 3, a shoulder alignment step 320 is conducted. As shown in FIG. 5, front shoulder line 415 is created from front shoulder end points 415a and 415b and back shoulder line 420 is created from back shoulder end points 420a and 420b, followed by identification of shoulder line center point 425. Referring to FIG. 6, front and rear shoulder line ends points 415a, 415b, 420a, 420b are then aligned using angle theta to rotate front joint image 405 and back joint image to create an aligned joint map (not shown). Information from the aligned joint map is then used to create an aligned body image depth map 700 as illustrated in FIG. 7.

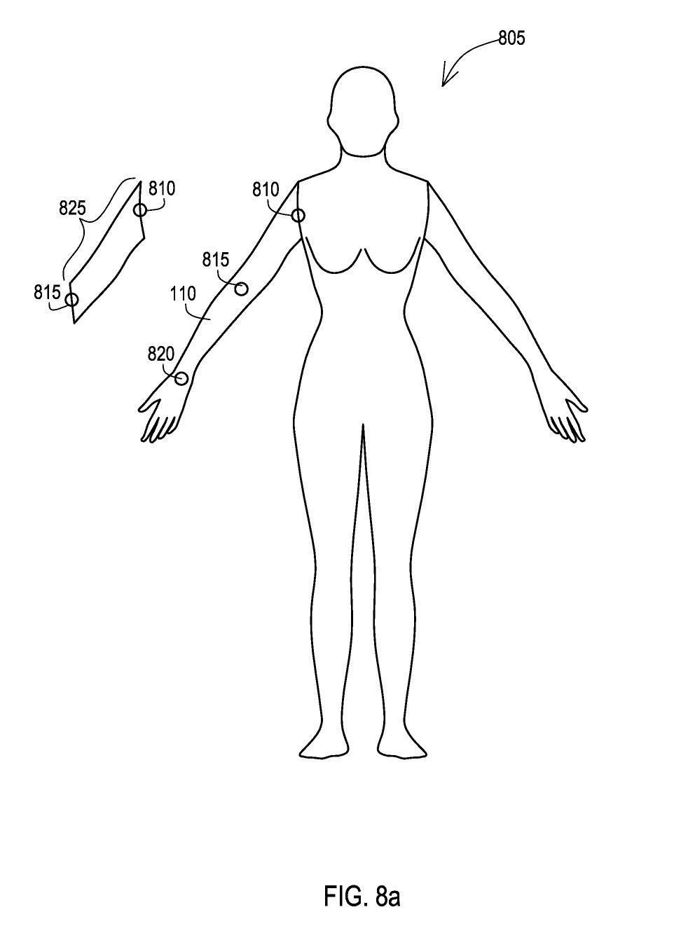

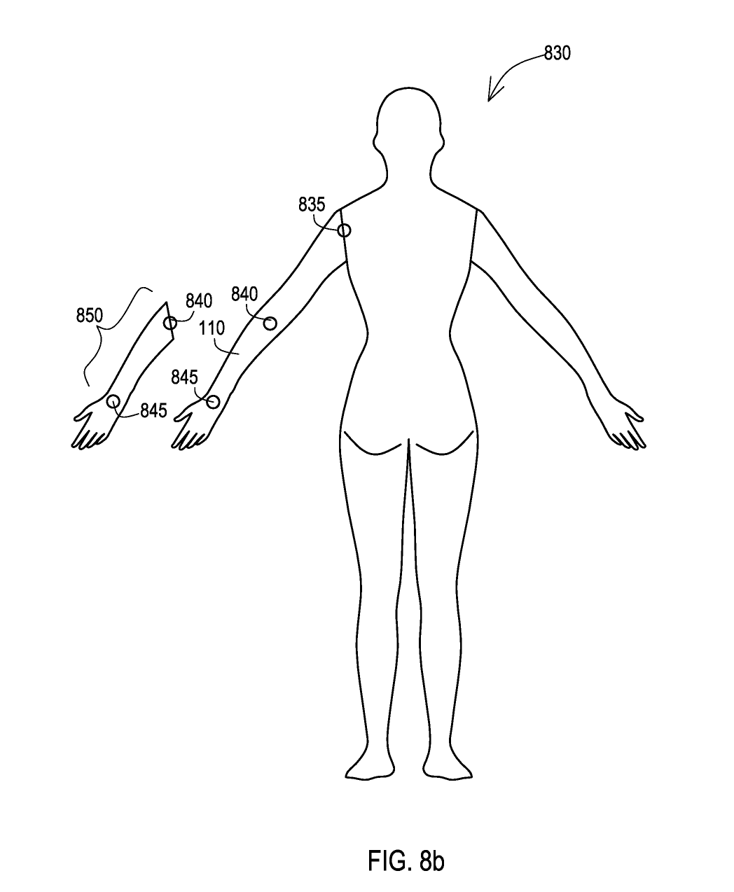

In step 325 of FIG. 3, an arm identification step 325 is conducted. Referring to FIG. 7, right arm 110 can be isolated from body image depth map 700 using the joint map information generated previously. As shown in FIG. 8a, front shoulder location 810 and front elbow location 815 are identified in front depth map 805 to allow identification of upper arm section 825. FIG. 8b illustrates back view 830. Either or both of front shoulder location 810 or back shoulder location 835 (FIG. 8b) and either or both of front elbow location 815 and 840 (FIG. 8b) can be used to identify upper arm section 825. Similarly, identification of front wrist location 820 and/or back wrist location 845 (FIG. 8b) allows identification of lower arm section 850 (FIG. 8b).

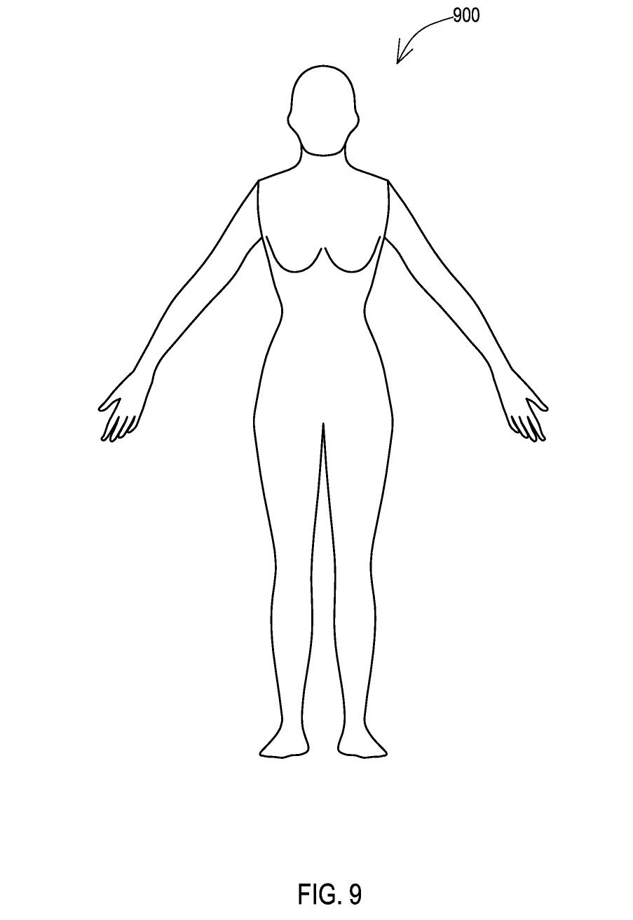

In step 330 of FIG. 3, the arms are aligned, in part, using the information generated in the arm identification step 325. As shown in FIG. 9, an aligned body image 900 is then provided from which subsequent steps can be conducted.

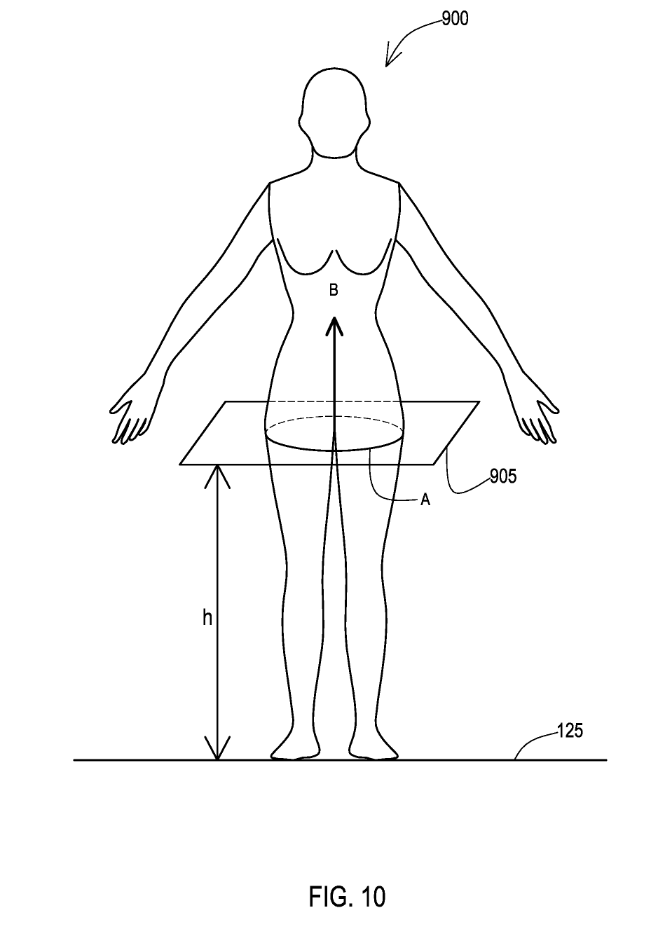

In step 335 of FIG. 3, 3D reconstruction of the patient 100 is provided. As illustrated in FIG. 10, aligned patient body image 900 is provided with a plane 905 that bisects the aligned body image 900 at approximately the hip location A, which can be identified as height h from floor 125. In other aspects, at least one plane 1005 parallel to the floor can bisect aligned body image 900 at another location (not shown), each of which will have its own height h relative to the floor 125.

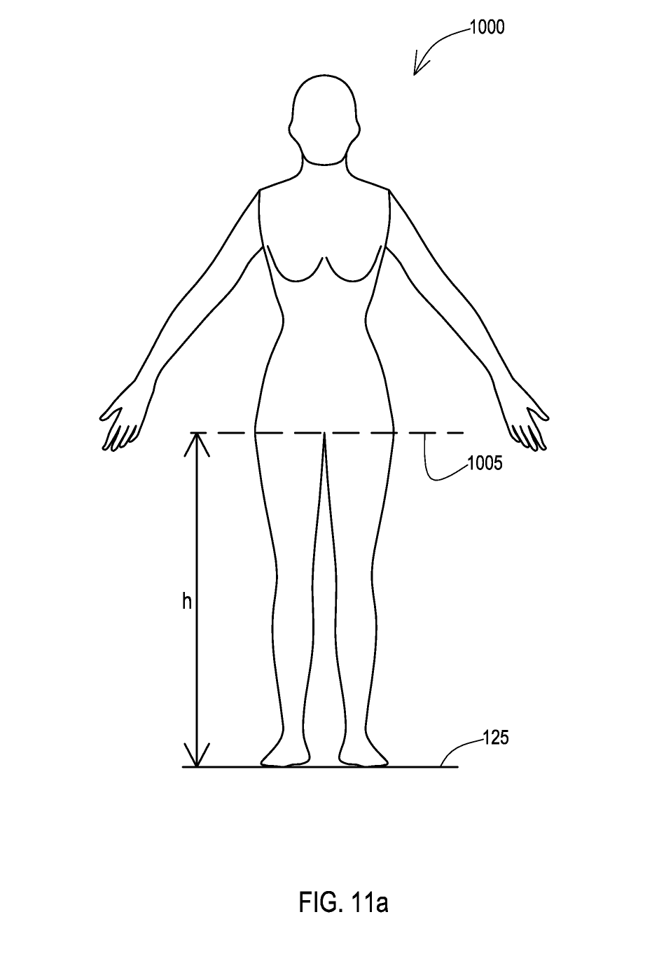

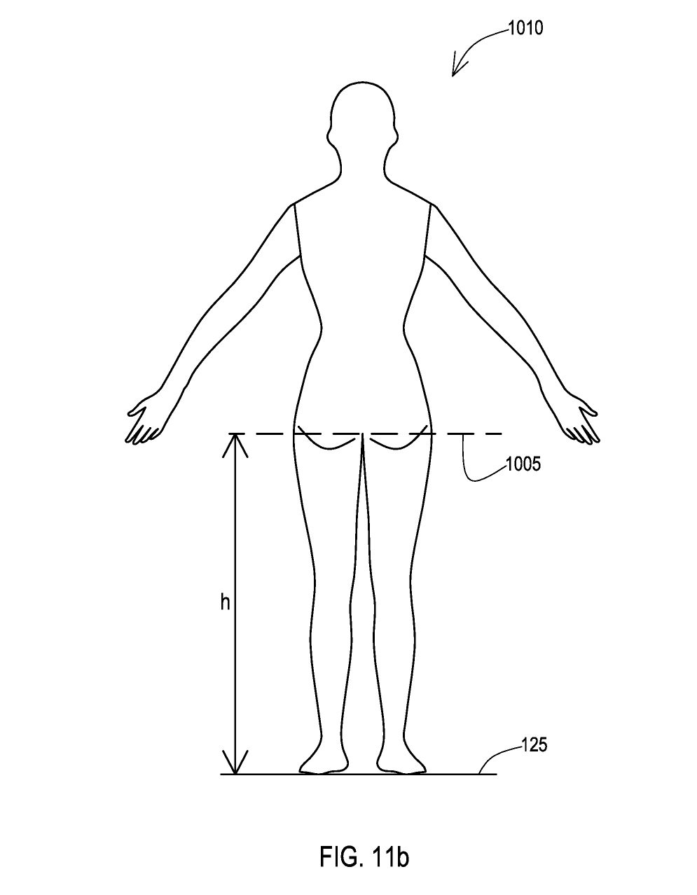

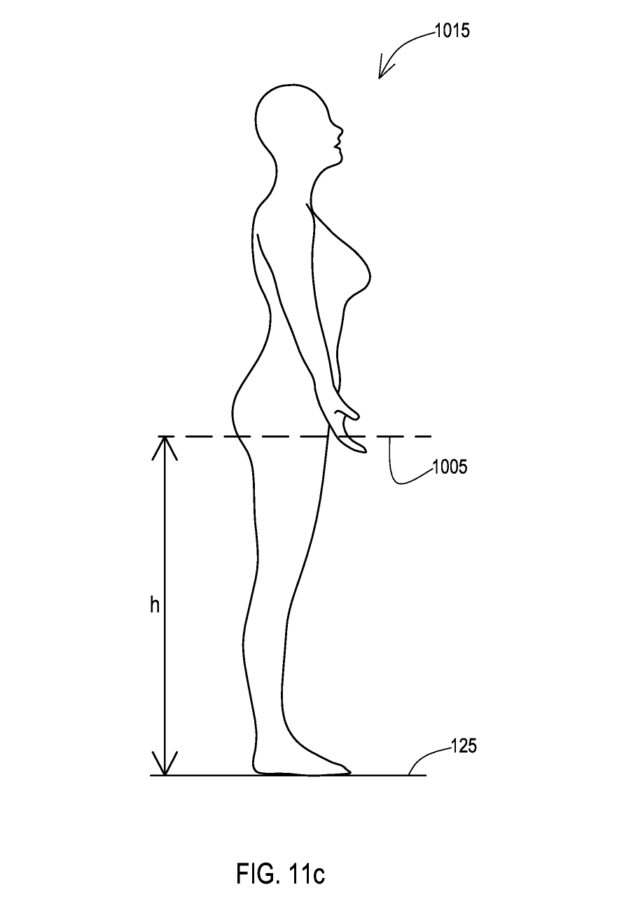

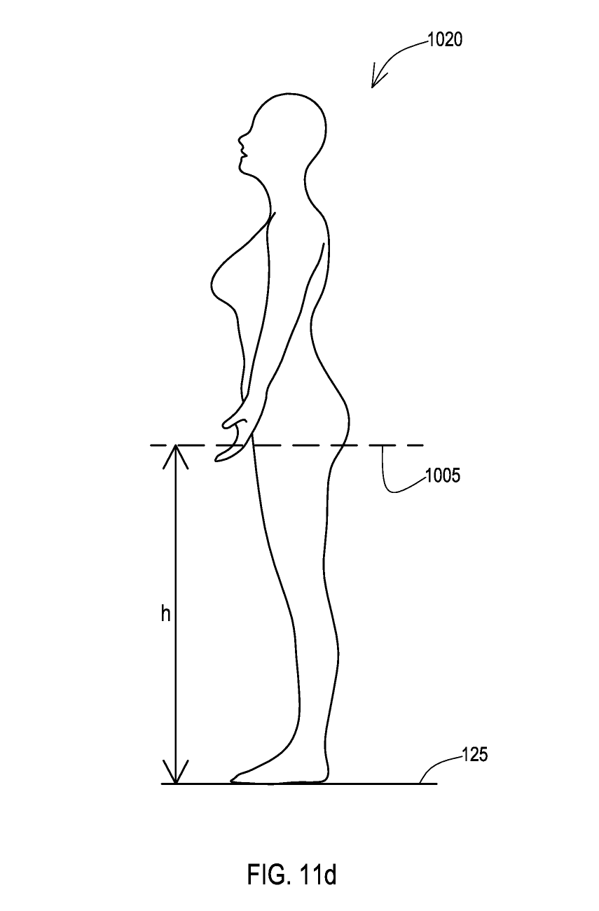

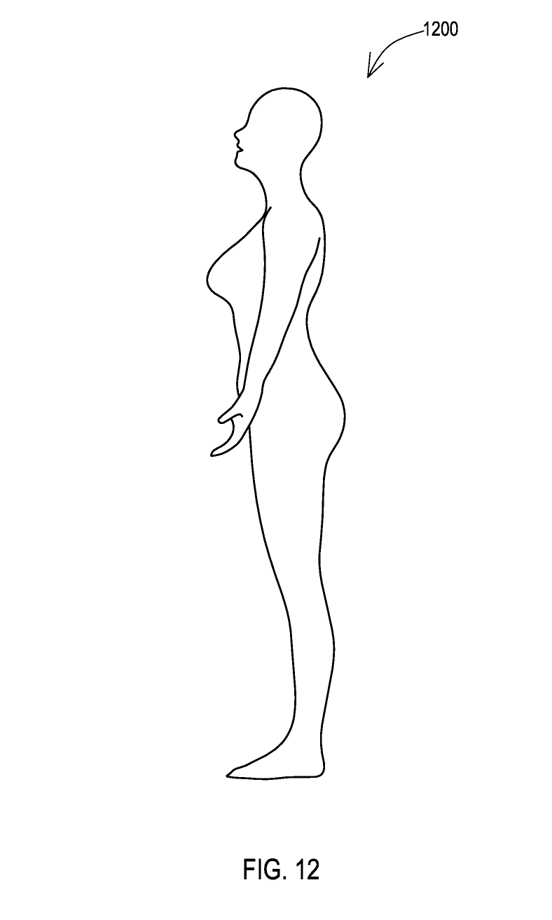

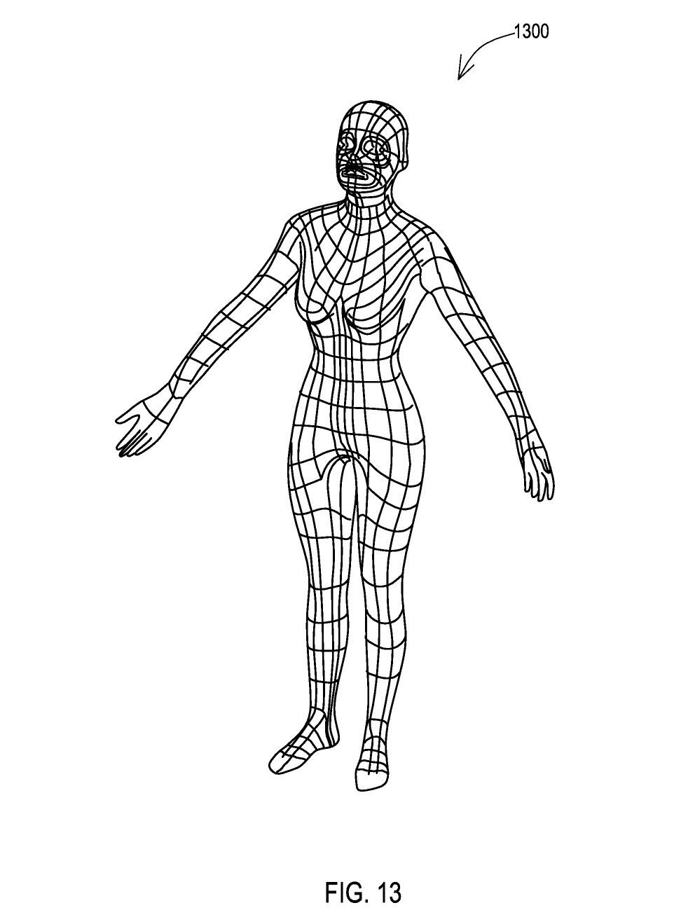

As shown in FIGS. 11a, 11b, 11c and 11d, height h is mapped as a line 1005 on each of the front, back right and left body image projections 1000, 1010, 1015 and 1020, respectively. The plurality of left and right images generated from the ToF imaging device (not shown) are analyzed to obtain the side distance of the patient as extracted from the actual patient measurement data so obtained, thereby providing a patient image 1200 having side dimensions approximately equal to that of the actual patient 100. FIG. 13 shows a 3D surface mesh 1300 that is a graphical representation of patient 100 as derived from the images generated from the above-described steps.

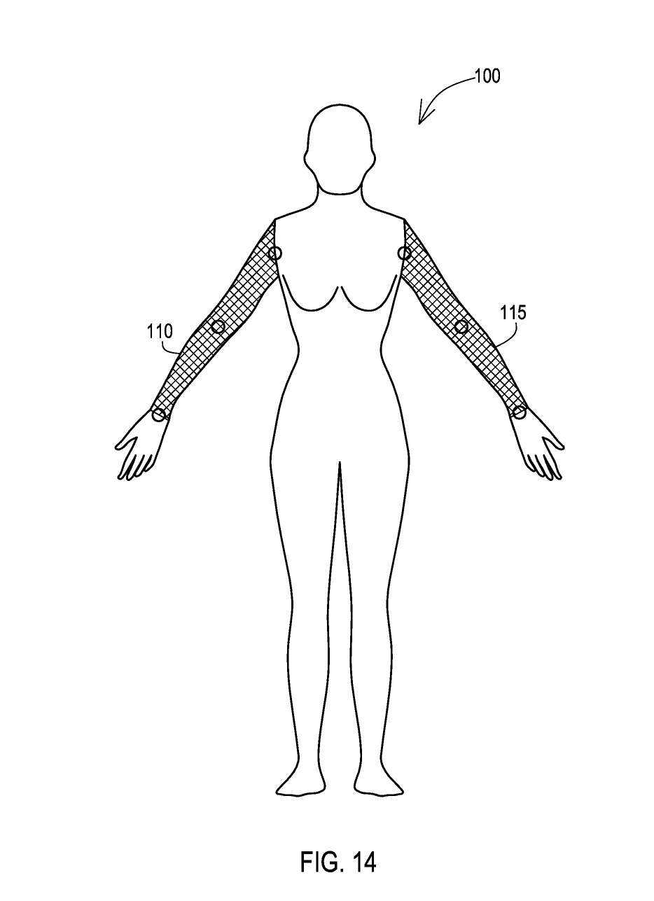

Referring now to step 340 of FIG. 3, from the 3D reconstruction process 335, anatomical measurements, in particular body part volume measurements of one or more of the patient's arms, can be generated in step 340. For example, as shown in FIG. 14, body part volume measurements or geometries can be generated of right arm 110 and/or left arm 115 of patient 100. Such body part volume measurements or geometries can be obtained using mathematical techniques well known to those of ordinary skill in the art. Such arm volume measurements can be used to detect, monitor and treat a patient for lymphedema as discussed in detail in the disclosure herewith.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A method comprising:
a. providing a patient in need of detection, monitoring, or treating for lymphedema, wherein the patient is positioned on a floor surface;
b. providing a time of flight (ToF) imaging device, wherein the imaging device is:
i. in operational communication with a computer system;
ii. configured to generate a plurality of images of the patient at from about 5 to about 50 frames per second; and
iii. positioned substantially stationary at a distance of from about 3 to about 8 feet from the patient and at a height of from about patient knee high to about patient shoulder high as measured relative to the patient;
c. directing the patient to position either or both of her arms or legs at a front imaging position, a left imaging position, a back imaging position, and a right imaging position, wherein, when the patient is in each imaging position the patient's arms or legs are each, independently, positioned at an angle of from about 10 to about 90 degrees as measured relative to a vertical line drawn through a center of the patient that is perpendicular to the floor surface;
d. generating, by the imaging device, a plurality of images of the patient when she is positioned in each of the front imaging position, the left imaging position, the back imaging position, and the right imaging position, wherein the images generated in each imaging position each, independently, comprise a plurality of front view patient images, left side view patient images, back side view patient images and right side view patient images; and
e. processing, by a computer, the plurality of patient images, thereby generating patient anatomical information about one or more of the patient's body parts.

2. The method of claim 1, wherein the generated patient anatomical information comprises measurement information of one or both of the patient's left and right arms or one or both of the patient's left and right legs.

3. The method of claim 2, further comprising generating at least one patient body part volume measurement from the generated patient measurement information, thereby providing at least one of a patient left arm volume measurement, a patient right arm volume measurement, a patient left leg volume measurement, or a patient right leg volume measurement.

4. The method of claim 3, wherein one or more of the at least one patient body part volume measurements are generated close in time to a treatment in which the patient undergoes a medical treatment involving disruption of the patient's lymph system.

5. The method of claim 4, wherein the medical treatment comprises a breast cancer treatment.

6. The method of claim 4, further comprising repeating steps a through e in one or more subsequent measurement events, thereby generating one or more of a subsequent patient left arm volume measurement, a subsequent patient right arm volume measurement, a subsequent patient left leg volume measurement or a subsequent patient right leg volume measurement.

7. The method of claim 6, further comprising the comparing the one or more of the first patient arm or leg volume measurements with the one or more subsequent arm or leg volume measurements, thereby determining whether the patient experiences a change in a corresponding arm or leg measurement volume over time.

8. The method of claim 7, further comprising determining the patient's weight substantially contemporaneously to generation of the one or more arm or leg volume measurements and to the one or more subsequent arm or leg volume measurements and, when a patient weight difference is detected between at least two of the volume measurements, generating a patient size differential.

9. The method of claim 8, wherein a diagnosis of lymphedema is provided when at least one of the subsequent patient arm or leg volume measurements is about 10% percent or more greater than one or more of the first arm or leg volume measurement of a corresponding arm or leg.

10. The method of claim 8, wherein a diagnosis of lymphedema is provided when at least one of the subsequent patient arm or leg volume measurements is about 5% percent or more greater than one or more of the first arm or leg volume measurement of a corresponding arm or leg.

11. The method of claim 9, wherein the corresponding arm or leg is the patient's contralateral arm or leg, thereby generating a comparison of an opposite patient arm or leg volume over a period of time.

12. The method of claim 1, wherein the plurality of front side view patient images, left side view patient images, back side view patient images and right side view patient images each, independently, comprises from about 20 to about 30 images of each patient view.

13. The method of claim 1, wherein the patient is positioned with her thumb in an upward position and her palm facing toward the imaging device when the plurality of images are generated.

14. The method of claim 1, wherein the image processing step includes:
a. a depth map generation step;
b. a joint identification step;
c. a first body part alignment step;
d. a body part identification step;
e. a second body part alignment step; and
f. a 3D reconstruction step.

15. The method of claim 2, further comprising deriving compression garment sizing dimensions from the patient arm or leg measurements, wherein the dimensions comprise a plurality of patient limb circumferences generated serially along a length of the patient's arm or leg.

16. The method of claim 1, further comprising deriving a first range of motion information from the generated patient arm or leg measurement information.

17. The method of claim 16, further comprising repeating steps a through e in one or more subsequent measurement events to generate one or more subsequent anatomical measurements from which subsequent range of motion information is derivable from the measurement information, wherein the first range of motion information is compared to the subsequent range of motion information, thereby generating patient range of motion comparison information.
